(19) United States
(12) Patent Application Publication
(10) Pub. No.: US 2023/0023223 A1
(43) Pub. Date: Jan. 26, 2023

(54) REMOTE DIAGNOSTIC SYSTEM WITH PORTABLE DIAGNOSTIC DEVICES AND METHOD THEREFOR

(71) Applicant: HIGHWAY INNOVATION INC., Calgary (CA)

(72) Inventors: Anja SLAMA, Calgary (CA); Imed JOUINI, Calgary (CA)

(21) Appl. No.: 17/871,759

(22) Filed: Jul. 22, 2022

Related U.S. Application Data
(60) Provisional application No. 63/225,293, filed on Jul. 23, 2021.

(51) Int. Cl. A61B 5/00; A61B 5/06

(52) U.S. Cl. CPC A61B 5/7465; A61B 5/0022; A61B 5/0024; A61B 5/7264; A61B 5/7275; A61B 5/06; A61B 5/7221; A61B 2560/0223; A61B 2560/0252

(57) ABSTRACT

A system has a plurality of diagnostic sensors and a first computing device interconnected to the plurality of diagnostic sensors for: commanding the plurality of diagnostic sensors to collect diagnostic data from a patient, determining if each of the plurality of diagnostic sensors are positioned at respectively predefined body locations of the patient, combining the collected diagnostic data for analysis when all of the plurality of diagnostic sensors are positioned at respectively predefined body locations of the patient, and prompting the patient to reposition at least one of the plurality of diagnostic sensors when the at least one sensor is determined as being mis-positioned from the corresponding predefined body location.

### CROSS-REFERENCE TO RELATED APPLICATIONS

This application claims the benefit of U.S. Provisional Patent Application Ser. No. 63/225,293 filed Jul. 23, 2022, the content of which is incorporated herein by reference in its entirety.

### FIELD OF THE DISCLOSURE

The present disclosure relates generally to a remote diagnostic system and method, and in particular to a remote diagnostic system with portable diagnostic devices and method for remote, real-time diagnosis.

### BACKGROUND

Conventionally, a patient needs to meet a doctor or physician in person for medical diagnosis and health advices. However, the majority of the clinic visits are for routine follow-up or minor complaints that only require general medical diagnosis. For example, according to the National center of Health Statistic of the United States, the primary reason for clinic visits is general medical diagnosis.

During a typical clinic visit to the doctor or physician, a visual examination may be performed for a preliminary diagnosis, for example, by inspecting throat, listening to the heart, lung, and abdomen with a stethoscope. Blood-pressure monitor and oximeter may also be used to better understand the patient's health status and conductions.

While the duration of most clinic visits is brief, such visits in total may consume a significant amount of resources and time of the clinics which may be otherwise used for other valuable health and medical tasks. Moreover, such clinic visits for routine follow-up or minor complaints are also a burden to patients.

Remote healthcare monitoring and telemedicine are known. In the United States, Kaiser Permanente reported in 2015 a 52% of the 110 million physician-patient interactions through remote healthcare monitoring, and the 2015 Canadian Telehealth Report that there were 411,778 telehealth clinical sessions in 2014. Based on the report from the Telehealth Forum on Oct. 5, 2017, patients and family “saved an estimated six million kilometres of travel for clinical services in 2016/17. According to University of Saskatchewan College of Nursing, eHealth Saskatchewan saw a 49% growth in patients using telehealth for clinical services between 2016 and 2017, including a 132% growth in patients seen in First Nations communities.” According to Ontario Telemedicine Networking (OTN), a total of 237,221,884 kilometers (km) of patient travel was avoided because of the use of telemedicine.

In a remote healthcare monitoring session, a physician uses a computer network to communicate with a patient and may perform diagnosis to some extent. The physician may then prescribe medication for the patient (the so called “telemedicine”, also called “telehealth”, “digital medicine”, “e-health”, or “mobile-health” (“m-health”)).

While remote healthcare monitoring and telemedicine are becoming more and more popular, the still have disadvantages. For example, the doctor and the patient have to travel to telemedicine-stations for remote diagnosis, or the system may require deployment of expensive home-based medical devices to patients' homes which may not be affordable to general public. Moreover, the repair cost of home-based medical devices (which include various testing monitors) is usually high even if only one or two sensors are damaged.

Although portable diagnostic devices are available in the market, such portable diagnostic devices usually need a doctor or a nurse to run the devices and collect the data for further analysis. On the other hand, existing home-based health-monitoring devices are either single-function devices that cannot be combined with other health-monitoring devices, or multifunction devices (such as wearable health-monitoring devices) which may cause a problem for the patient and the doctor to diagnostic different areas of the body at the same time.

Therefore, there is a desire for a novel portable diagnostic device and system with low cost, ease of use by nonmedical people for remote, real-time diagnosis.

### SUMMARY

According to one aspect of this disclosure, there is provided a system comprising: a plurality of diagnostic sensors; and a first computing device interconnected to the plurality of diagnostic sensors for: commanding the plurality of diagnostic sensors to collect diagnostic data from a patient; determining if each of the plurality of diagnostic sensors are positioned at respectively predefined body locations of the patient; combining the collected diagnostic data for analysis when all of the plurality of diagnostic sensors are positioned at respectively predefined body locations of the patient; and prompting the patient to reposition at least one of the plurality of diagnostic sensors when the at least one sensor is determined as being mis-positioned from the corresponding predefined body location.

In some embodiments, the system further comprises a camera, wherein the first computing device is interconnected with the camera for audio and video communication with the patient.

In some embodiments, the first computing device is configured to calibrate the plurality of diagnostic sensors.

In some embodiments, the first computing device is configured to verify the collected diagnostic data.

In some embodiments, the plurality of diagnostic sensors comprises a blood pressure monitor.

In some embodiments, the plurality of diagnostic sensors comprises an oximeter.

In some embodiments, the plurality of diagnostic sensors comprises a temperature sensor.

In some embodiments, the plurality of diagnostic sensors comprises an electrical stethoscope.

In some embodiments, the plurality of diagnostic sensors comprises a heartbeat monitor.

In some embodiments, the plurality of diagnostic sensors comprises a high-definition otoscope.

In some embodiments, the system further comprises an ambient temperature sensor for measuring ambient temperature proximate the patient.

In some embodiments, the system is portable.

In some embodiments, the system further comprises one or more positioning components to determine a geographical location of the first computing device.

In some embodiments, the system further comprises an output device for displaying information.

In some embodiments, the first computing device is further configured for: sending at least one of the collected diagnostic data and the combined diagnostic data to a second computing device via a network.

In some embodiments, said sending the at least one of the collected diagnostic data and the combined diagnostic data to the second computing device via the network comprises: sending in real-time the at least one of the collected diagnostic data and the combined diagnostic data to the second computing device via the network.

In some embodiments, the second computing device is configured for processing the collected diagnostic data and the combined diagnostic data into processed diagnostic data, and the second computing device comprises an artificial intelligence (AI) model for predicting health conditions of the patient using the processed diagnostic data.

In some embodiments, the second computing device is configured to train the AI model using the processed diagnostic data.

In some embodiments, the second computing device is configured to encrypt the collected diagnostic data, the combined diagnostic data, the processed diagnostic data, and the predicted health conditions.

According to one aspect of this disclosure, there is provided a method comprising the steps of: collecting diagnostic data from a patient using a plurality of diagnostic sensors; determining if all of the plurality of diagnostic sensors are positioned at respectively predefined body locations of the patient; combining the collected diagnostic data for analysis when all of the plurality of diagnostic sensors are positioned at respectively predefined body locations of the patient; and prompting the patient to reposition at least one of the plurality of diagnostic sensors when the at least one sensor is determined as being mis-positioned from the corresponding predefined body location.

In some embodiments, the method further comprises the step of turning on a camera, and wherein the step of prompting the patient comprises communicating with the patient with audio and video.

In some embodiments, the method further comprises the step of calibrating at least one of the diagnostic sensors.

In some embodiments, the method further comprises the step of verifying the diagnostic data.

In some embodiments, the step of collecting diagnostic data comprises collecting a non-invasive blood pressure.

In some embodiments, the step of collecting diagnostic data comprises collecting a peripheral oxygen saturation level.

In some embodiments, the step of collecting diagnostic data comprises collecting a body temperature.

In some embodiments, the step of collecting diagnostic data comprises collecting an electrical stethoscope measurement.

In some embodiments, the step of collecting diagnostic data comprises collecting an electrocardiogram.

In some embodiments, the step of collecting diagnostic data comprises collecting a high-definition otoscope reading.

In some embodiments, the method further comprises the step of measuring ambient temperature of the patient's environment.

In some embodiments, the method further comprises the step of determining a geographical location of the system.

In some embodiments, the method further comprises the step of sending at least one of the collected diagnostic data and the combined diagnostic data to a computing device via a network.

In some embodiments, the step of sending the collected diagnostic data comprises sending in real-time the at least one of the collected diagnostic data and the combined diagnostic data to the computing device via the network.

In some embodiments, the method further comprises processing the collected diagnostic data and the combined diagnostic data into processed diagnostic data; and predicting health conditions of the patient using an AI model and the processed diagnostic data.

In some embodiments, the method further comprises the step of training of the AI model using the processed diagnostic data.

In some embodiments, the method further comprises the step of encrypting the collected diagnostic data, the combined diagnostic data, the predicted health conditions, and the predicted health conditions.

### BRIEF DESCRIPTION OF THE DRAWINGS

FIG. 1 is a schematic diagram of remote diagnostic system, according to some embodiments of this disclosure;

FIG. 2 is a schematic diagram showing a simplified hardware structure of the remote diagnostic system shown in FIG. 1;

FIG. 3 a schematic perspective view of an electrical stethoscope of the remote diagnostic system shown in FIG. 1;

FIG. 4 a schematic diagram showing a simplified software architecture of the remote diagnostic system shown in FIG. 1;

FIG. 5 is a block diagram showing the dataflow of the remote diagnostic system shown in FIG. 1;

FIG. 6 is a flowchart showing a process executed by the remote diagnostic system shown in FIG. 1 for performing remote diagnosis and telemedicine, according to some embodiments of this disclosure;

FIG. 7 is a flowchart showing a process executed by the remote diagnostic system shown in FIG. 1 for performing remote diagnosis and telemedicine, according to some other embodiments of this disclosure;

FIGS. 8 and 9 show exemplary screen images of a client computing device of a physician in the remote diagnostic system shown in FIG. 1 for visual diagnosis and for helping a patient to position a sensor at a proper body position; and

FIGS. 10 and 11 show exemplary screen images of a diagnostic hub of the remote diagnostic system shown in FIG. 1 for displaying diagnostic data collected by various portable diagnostic devices of the remote diagnostic system shown in FIG. 1.

### DETAILED DESCRIPTION

Embodiments disclosed herein relate to a remote diagnostic system and method. The remote diagnostic system comprises a plurality of portable diagnostic devices each having various sensors for attaching to various body locations of a patient for collecting diagnostic data. The portable diagnostic devices may be connected with a computer server directly through a network. Alternatively, the portable diagnostic devices may be connected to a diagnostic hub which is in turn connected to the computer server directly through the network.

The diagnostic data collected by the sensors of the portable diagnostic devices may be combined for detecting mis-positioning of one or more sensors, calibrating one or more sensors, and/or indicating errors and/or failures of one or more sensors. The combined diagnostic data is securely transmitted to the server computer for analysis by an artificial intelligence (AI) engine using deep learning. The combined diagnostic data and analytic results are stored in a secure database of the server computer (with encryption as needed) and are sent to a patient-designated physician for evaluating the patient's health conditions. The physician may then provide a prescription and health advices to the patient through the remote diagnostic system.

After attaching the sensors of the portable diagnostic devices to the patient's body, the portable diagnostic devices may collect diagnostic data synchronously and in a continuous manner, and the collected diagnostic data may be transmitted to the server computer in real-time, thereby enabling accurate, continuous, prompt, and on-demand diagnostic data collection, analysis, and evaluation. Of course, a patient may only use a subset of the portable diagnostic devices as the physician advises or as needed.

In some embodiments, the portable diagnostic devices may include blood-pressure monitors, oximeters, temperature sensors, electrical stethoscopes, heartbeat monitor, high-definition otoscope, and/or the like for monitoring vital clinical parameters of the patient such as non-invasive blood pressure (NIBP), electrocardiogram (ECG), peripheral oxygen saturation (SpO₂) level, respiration, body temperature, stethoscope, otoscope, and/or the like.

In some embodiments, cameras are used and audio/video communications are established between physicians and patients for visual diagnosis and for instructing the patients to position sensors at proper body locations.

By using the remote diagnostic system, face-to-face physician-patient interactions may be significantly reduced.

### System Overview and Hardware Structure

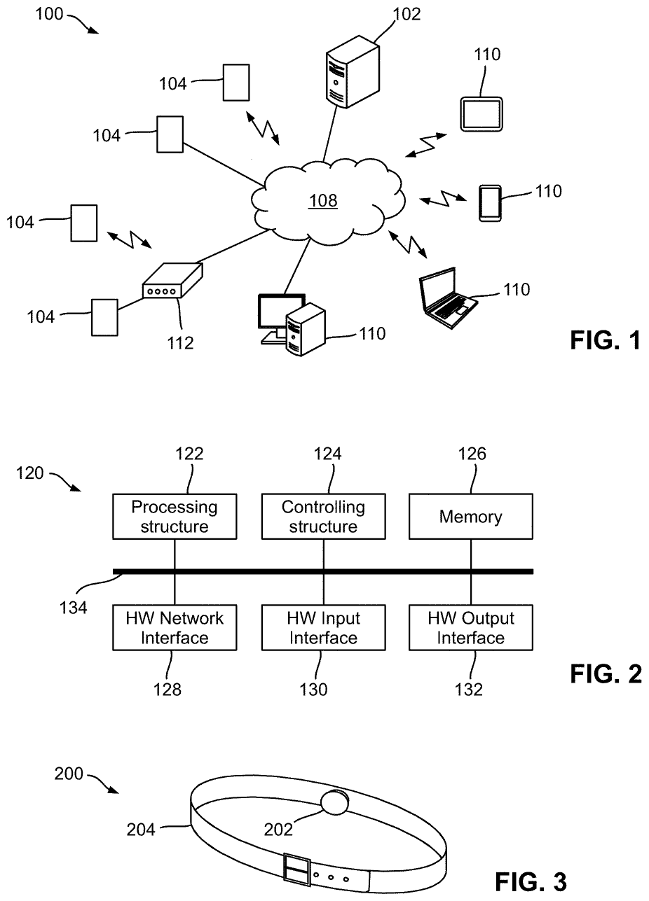

Turning now to FIG. 1, a diagnostic system according to some embodiments of this disclosure is shown and is generally identified using reference numeral 100. As shown, the diagnostic system 100 may be a LIFEBOX™ diagnostic system (LIFEBOX is a trademark of Highway Innovation Inc. of Calgary, Alberta, Canada) and comprises at least one server computer 102, a plurality of portable diagnostic devices 104, and a plurality of client computing devices 110 operated by physicians and patients, all functionally interconnected by a network 108, such as the Internet, a local area network (LAN), a wide area network (WAN), a metropolitan area network (MAN), and/or the like, via suitable wired and/or wireless networking connections. In various embodiments, the portable diagnostic devices 104 may be directly connected to the network 108 for communicating with the server computer 102 or through a diagnostic hub 112 via suitable wired and/or wireless networking connections.

The portable diagnostic devices 104 collect diagnostic data (also called “sensor data” hereinafter) from associated patients and transmit collected diagnostic data to the server computer 102. The server computer 102 analyzes the diagnostic data and transmits the analytical results to the client computing devices 110 operated by physicians. The physicians may prepare prescription and provide health advices based on the received analytical results. The physicians' prescriptions and health advices are then transmitted from the client computing devices 110 operated by physicians to the client computing devices 110 operated by patients via the network 108 and the server computer 102.

In some embodiments, the server computer may also transmit analytical results to the client computing devices 110 operated by the patients.

In some embodiments, the physicians' prescriptions may also be transmitted to a client computer device of a pharmacist.

The server computer 102 may be a computing device designed specifically for use as a server, or a general-purpose computing device acting the server computer while also being used by a user (for example, a physician or a patient). The server computer 102 may execute one or more server programs.

The client computing devices 110 may be portable and/or non-portable computing devices such as laptop computers, tablets, smartphones, Personal Digital Assistants (PDAs), desktop computers, and/or the like. Each client computing devices 110 may execute one or more client application programs which sometimes may be called “apps”.

Generally, the computing devices 102 and 110 have a similar hardware structure such as a hardware structure 120 shown in FIG. 2. As shown, the computing device 102 or 110 comprises a processing structure 122, a controlling structure 124, one or more non-transitory computer-readable memory or storage devices 126, a network interface 128, a hardware input interface 130, and a hardware output interface 132, functionally interconnected by a system bus 134. The computing device 102 or 110 may also comprise other components 134 coupled to the system bus 134.

The processing structure 122 may be one or more single-core or multiple-core computing processors such as INTEL® microprocessors (INTEL is a registered trademark of Intel Corp., Santa Clara, Calif., USA), AMD® microprocessors (AMD is a registered trademark of Advanced Micro Devices Inc., Sunnyvale, Calif., USA), ARM® microprocessors (ARM is a registered trademark of Arm

Ltd., Cambridge, UK) manufactured by a variety of manufactures such as Qualcomm of San Diego, Calif., USA, under the ARM® architecture, or the like. When the processing structure 122 comprises a plurality of processors, the processors thereof may collaborate via a specialized circuit such as a specialized bus or via the system bus 134.

The processing structure 122 may also comprise one or more real-time processors, programmable logic controllers (PLCs), microcontroller units (MCUs), μ-controllers (UCs), specialized/customized processors and/or controllers using, for example, field-programmable gate array (FPGA) or application-specific integrated circuit (ASIC) technologies, and/or the like.

Generally, each processor of the processing structure 122 comprises necessary circuitries implemented using technologies such as electrical and/or optical hardware components for executing one or more programs in the form of software or firmware, as the design purpose and/or the use case maybe, to perform various tasks. A processor may also comprise “hard-wired” circuitry for performing predefined tasks.

The controlling structure 124 comprises one or more controlling circuits, such as graphic controllers, input/output chipsets and the like, for coordinating operations of various hardware components and modules of the computing device 102 or 110.

The memory 126 comprises one or more storage devices or media accessible by the processing structure 122 and the controlling structure 124 for reading and/or storing instructions for the processing structure 122 to execute, and for reading and/or storing data, including input data and data generated by the processing structure 122 and the controlling structure 124. The memory 126 may be volatile and/or non-volatile, non-removable or removable memory such as RAM, ROM, EEPROM, solid-state memory, hard disks, CD, DVD, flash memory, or the like. In use, the memory 126 is generally divided into a plurality of portions for different use purposes. For example, a portion of the memory 126 (denoted as storage memory herein) may be used for long-term data storing, for example, for storing files or databases. Another portion of the memory 126 may be used as the system memory for storing data during processing (denoted as working memory herein).

The network interface 128 comprises one or more network modules for connecting to other computing devices or networks through the network 110 by using suitable wired or wireless communication technologies such as Ethernet, WI-FI® (WI-FI is a registered trademark of Wi-Fi Alliance, Austin, Tex., USA), BLUETOOTH® (BLUETOOTH is a registered trademark of Bluetooth Sig Inc., Kirkland, Wash., USA), Bluetooth Low Energy (BLE), Z-Wave, Long Range (LoRa), ZIGBEE® (ZIGBEE is a registered trademark of ZigBee Alliance Corp., San Ramon, Calif., USA), wireless broadband communication technologies such as Global System for Mobile Communications (GSM), Code Division Multiple Access (CDMA), Universal Mobile Telecommunications System (UMTS), Worldwide Interoperability for Microwave Access (WiMAX), CDMA2000, Long Term Evolution (LTE), 3GPP, 5G New Radio (5G NR) and/or other 5G networks, and/or the like. In some embodiments, parallel ports, serial ports, USB connections, optical connections, or the like may also be used for connecting other computing devices or networks although they are usually considered as input/output interfaces for connecting input/output devices.

The hardware input interface 130 comprises one or more input components for interconnecting with input devices for receiving data therefrom. Examples of input devices may be touch-sensitive screen, touch-sensitive whiteboard, touch-pad, keyboards, computer mouse, trackball, microphone, scanners, cameras (including webcams), and/or the like. An input device may be a physically integrated part of the computing device 102 or 110 (for example, the touch-pad of a laptop computer or the touch-sensitive screen of a tablet), or may be a device physically separate from, but functionally coupled to, other components of the computing device 102 or 110 (for example, a computer mouse). Moreover, an input device, in some implementation, may be integrated with an output interface to form an integrated input/output device such as a touch-sensitive screen or touch-sensitive whiteboard.

The hardware output interface 132 comprises one or more output components for interconnecting with output devices for outputting data thereto. Examples of output devices may be displays (such as monitors, LCD displays, LED displays, projectors, and the like), speakers, printers, virtual reality (VR) headsets, augmented reality (AR) goggles, and/or the like. The output device 132 may be a physically integrated part of the computing device 102 or 110 (for example, the display of a laptop computer or tablet), or may be a device physically separate from but functionally coupled to other components of the computing device 102 or 110 (for example, the monitor of a desktop computer).

The system bus 134 interconnects various components 122 to 132 enabling them to transmit and receive data and control signals to and from each other.

Although now shown, the computing device 102 or 110 may also comprise other components such as one or more positioning components, temperature sensors, barometers, inertial measurement unit (IMU), and/or the like. Examples of the positioning components may be one or more global navigation satellite system (GNSS) components (e.g., one or more components for operation with the Global Positioning System (GPS) of USA, Global'naya Navigatsionnaya Sputnikovaya Sistema (GLONASS) of Russia, the Galileo positioning system of the European Union, and/or the Beidou system of China).

Referring again the FIG. 1, the diagnostic hub 112 may be a customized computing device or a computing device similar to the client computing device 110 (for example, a laptop computer, a desktop computer, a tablet, or a smartphone) with suitable hardware and software device interfaces for interconnecting with the portable diagnostic devices 104. The diagnostic hub 112 may execute one or more application programs for combining the diagnostic data and using the combined diagnostic data to calibrate the portable diagnostic devices 104.

In some embodiments, the diagnostic hub 112 may also be a client computing device 110.

The hardware structure of the diagnostic hub 112 may be similar to the hardware structure 120. Specifically, the hardware input interface 130 and hardware output interface 132 thereof comprise input and output components for interconnecting with the portable diagnostic devices 104 via suitable wired connections such as parallel ports, serial ports, USB connections, optical connections, and/or the like, or via suitable wireless connections such as BLUETOOTH®, BLE, Z-Wave, LoRa, ZIGBEE®, and/or the like. The controlling structure 124 is used for controlling the sensors of the portable diagnostic devices 104 for data collection. The processing structure 122 executes application programs for processing and combining collected data and/or performing some data analysis.

In some embodiments, the diagnostic hub 112 may also comprise one or more positioning components for providing the patient's location.

In some embodiments, the diagnostic hub may further comprise a temperature sensor for determining the ambient temperature data during the diagnostic to help physician to better interpret the bio-signals.

In some embodiments, the hardware structure of the diagnostic hub 112 may be a simplified version thereof. For example, the diagnostic hub 112 in some embodiments may not comprise any display and only comprise one or more indicator lights for indicating the operation of the diagnostic hub 112. As another example, the processing structure 122 and controlling structure 124 of the diagnostic hub 112 in some embodiments may be implemented as one component (for example, as one circuit) for performing sensor control and some data processing/combination tasks. As a further example, the diagnostic hub 112 in some embodiments may not comprise a system bus 134 and the components 122 to 132 of the diagnostic hub 112 may be interconnected via separate circuits.

In some embodiments, the diagnostic hub 112 may comprise a 12 volts (V), 2 amps (A), alternate-current (AC) adaptor with an internal rechargeable battery enabling the patient to use at home or in travel. The diagnostic hub 112 may also comprise a network interface 128 for connecting to the network 108 using Ethernet, WI-FI®, or wireless broadband communication technologies. The diagnostic hub 112 may also use a short-range wireless communication technologies such as BLUETOOTH® to connect to a network access point or so-called hot spot to connect to the network 108. The diagnostic hub 112 may further comprise a 7″ (that is 7 inches) touch-screen with a resolution of 1024 by 600 pixels and a low response time, making the finger-touch or stylus-touch easer and allowing instantaneous transition between menu and navigate through sensors interfaces.

In some embodiments, the portable diagnostic devices 104 may comprise individual diagnostic devices each having necessary sensors attachable to a suitable location (and preferably the best location for collecting diagnostic data) of a patient for collecting therefrom a specific diagnostic data item (for example, blood pressure). In some other embodiments, the portable diagnostic devices 104 may comprise multifunctional diagnostic devices with various types of sensors attachable to various suitable locations (and preferably various best locations for collecting diagnostic data) of the patient for collecting therefrom various diagnostic data items (for example, blood pressure, body temperature, and the like). For example, the portable diagnostic devices 104 may comprise:
camera;
blood-pressure monitor;
oximeter;
temperature sensor;
electrical stethoscope;
heartbeat monitor; and
high-definition otoscope.

In some embodiments, the camera may be a high-definition (HD) webcam for a physician to conduct remote visual diagnosis and for the doctor to instruct the patient to perform diagnostic steps and/or how to use a portable diagnostic device 104.

The blood-pressure monitor may be in the form of a blood-pressure cuff for attaching to a patient's upper arm (for example, at the biceps zone thereof) to create a hyperbaric chamber thereabove for measuring the patient's blood pressure such as the NIBP. The blood-pressure monitor may be connected to the diagnostic hub 112 through a suitable wired or wireless connection such that the sensor (for example a sphygmomanometer) of the blood-pressure monitor is controlled by the diagnostic hub 112 for real-time blood-pressure measurement.

In some embodiments, the blood-pressure monitor may measure systolic and diastolic pressures with the following sensor parameters:
systolic blood pressure: 60 millimeters of mercury (mmHg) to 250 mmHg;
diastolic blood pressure: 30 mmHg to 180 mmHg;
static pressure accuracy: ±3 mmHg or 2%, whichever is greater.

The blood-pressure monitor may have other suitable sensor parameters in other embodiments.

The oximeter is used for determining the oxygen saturation of the patient's blood (such as the peripheral oxygen saturation (SpO₂) level). In some embodiments, the oximeter comprises a finger-clip oxygen sensor for attaching to the patient's finger or toe. This sensor uses a light source to emit red and/or infrared (IR) light through blood-perfused tissue and uses a photodetector to detect the reflected light. The oximeter measures the coefficient of the concentration of the oxygenated hemoglobin (HbO2) and the hemoglobin (Hb) through measurement of the reflected light for determining the absorption of the light in the tissue of the patient and then the arterial oxygen saturation.

The oximeter may be connected to the diagnostic hub 112 through a suitable wired or wireless connection such that the finger-clip oxygen sensor of the oximeter is controlled by the diagnostic hub 112 for real-time oxygen-saturation measurement.

In some embodiments, the oximeter may measure the oxygen saturation with the following sensor parameters:
measurement range: 35 to 100; and
accuracy: ±2% (for the percentage of oxygen in the blood within 80% to 100%), ±3% (for the percentage of oxygen in the blood within 70% to 79%), not specified (that is, low accuracy; for the percentage of oxygen in the blood within 35% to 69%).

The oximeter may have other suitable sensor parameters in other embodiments.

In some embodiments, the temperature sensor may be a contact thermometer with negative temperature coefficient (NTC) and high precision for measuring the patient's body temperature, with a measurement range of 25° C. to 45° C. and an accuracy of ±0.2° C. The temperature sensor may have other suitable measurement range and accuracy in other embodiments.

In some other embodiments, the temperature sensor may be an IR thermometer for measuring the patient's body temperature without physical contact. The temperature sensor detects the intensity of IR radiation naturally emanated from the patient's body surface.

The temperature measurement may be classified as below:
hypothermia: <35.0° C. (that is, 95.0° F.);
normal: 36.5° C. to 37.5° C. (that is, 97.7° F. to 99.5° F.);
fever: >37.5° C. to 38.3° C. (that is, 99.5° F. to 100.9° F.); and
hyperpyrexia: >40.0° C. to 41.5° C. (that is, 104° F. to 106.7° F.).

As shown in FIG. 3, in some embodiments, the electrical stethoscope 200 may comprise a digital stethoscope sensor 202 mounted on an adjustable belt 204 for free movement surrounding the patient's body, for example, moving the belt 204 up or down, and/or rotating the belt 204 to position the digital stethoscope sensor 202 at front, side, or back of the patient's body, thereby allowing the patient to position the electrical stethoscope to hard-to-reach areas for self-diagnosis. The electrical stethoscope may have the following sensor parameters:
heart sounds range: 20 Hertz (Hz) to 230 Hz;
lung sound range: 100 Hz to 800 Hz; and
measurement of heart rate within a range of 30 beats per minute (bpm) to 300 bpm and a resolution of one bpm.

The electrical stethoscope 200 may have other suitable sensor parameters in other embodiments.

In some embodiments, the heartbeat monitor may be a five-lead ECG monitor with the following parameters:
ECG measurement range: 25 bpm to 250 bpm;
ECG measurement accuracy: ±2 bpm or 2%, whichever is greater;
respiratory rate (RR) measurement range: 5 breaths per minute (brpm) to 100 brpm; and
respiratory rate measurement accuracy: ±2 brpm.

The heartbeat monitor may have other suitable sensor parameters in other embodiments.

In some embodiments, the HD otoscope may comprise a HD camera with six 6 LED lights for examining the condition of the patient's nasal, ear canal, and/or eardrum with the following parameters:
resolution: 640 by 480 pixels;
focal length: 2 centimeters (cm); and
viewing angle: 70 degrees.

The HD otoscope may have other suitable sensor parameters in other embodiments.

As will be described below, the portable diagnostic devices 104 may continuously measure and collect in real-time the diagnostic data using the sensors thereof positioned at various body locations. The diagnostic data measurement and collection of various portable diagnostic devices 104 may be conducted in a synchronous and simultaneous manner to collect vital clinic parameters such as NIBP, SPO₂, ECG, body temperature, otoscope images, and/or the like. The collected diagnostic data may be combined to improve data accuracy, to calibrate the sensors of the portable diagnostic devices 104, and to warn the patient about errors and potential failures of one or more sensors.

### Software Structure and System Operation

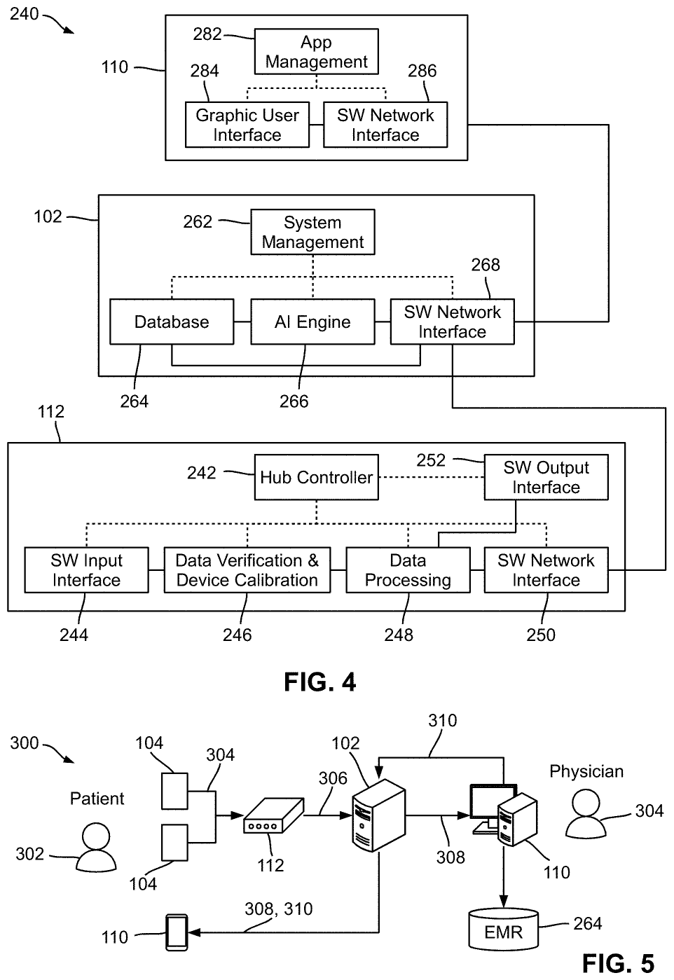

As those skilled in the art will appreciate, the diagnostic system 100 is a combination of hardware and software. FIG. 4 shows the software structure 240 of the diagnostic system 100 in some embodiments. FIG. 5 shows the dataflow 300 of the diagnostic system 100.

While not shown in FIG. 4, each of the server computer 102 and the client computing device 110 may comprise a suitable operating system for managing various hardware and software components thereof. The operating system also manages the communication with other computing devices via the network 108. As those skilled in the art will appreciate, the operating system may be any suitable operating system such as MICROSOFT® WINDOWS® (MICROSOFT and WINDOWS are registered trademarks of the Microsoft Corp., Redmond, Wash., USA), APPLE® OS X, APPLE® iOS (APPLE is a registered trademark of Apple Inc., Cupertino, Calif., USA), Linux, ANDROID® (ANDROID is a registered trademark of Google Inc., Mountain View, Calif., USA), or the like. The the server computer 102 and the client computing device 110 may all have the same operating system, or may have different operating systems.

Referring to FIGS. 4 and 5, the diagnostic hub 112 comprises a hub controller 242 controlling a software input interface 244, a data verification and device calibration module 246, a data processing module 248, a software network interface 250, and a software output interface 252.

Under the control of the hub controller 242, the software output interface 252 operates the hardware output interface 132 to instruct the portable diagnostic devices 104 to collect diagnostic data 304 from the patient 302. The software input interface 244 operates the hardware input interface 128 to communicate with the portable diagnostic devices 104 for receiving collected diagnostic data 304, and then transmits the received diagnostic data to the data verification and device calibration module 246. The data verification and device calibration module 246 verifies the correctness of the diagnostic data 304 and calibrate the portable diagnostic devices 104 as needed. After data verification, the verified diagnostic data is sent to the data processing module 248 for processing. The processed diagnostic data is then send to the software network module 250 which operates the hardware network module 128 for transmitting the processed diagnostic data 306 to the computer server 102. The processed diagnostic data may also be send to the software output interface 252 which operates the hardware output interface 132 to display some or all of the processed diagnostic data on the screen of the diagnostic hub 112.

The server computer 102 comprises a system management module 262 managing a secure database 264, an artificial intelligence (AI) engine 266 such as a deep-learning engine, and a software network interface 268. Under the control of the system management module 262, the software network interface 268 operates the hardware network module 128 of the server computer 102 to communicate with the software network module 250 of the diagnostic hub 112 for receiving the processed diagnostic data 306. The software network interface 268 then transmits the processed diagnostic data 306 to the AI engine 266, which applies the processed diagnostic data to an AI model for generating predictions 308 of patients' health conditions. The AI engine 266 also uses the processed diagnostic data to train the AI model. The health predictions 308 are then stored in the secure database 264 (with encryption as needed) and are sent to the client computing devices 102 (operated by physicians 304 and/or patients 302) via the software network interface 268.

The client computing device 110 comprises an app management module 282 managing a graphic user interface (GUI) 284 such as a web portal and a software network interface 286. Under the control of the app management module 282, the software network interface 286 receives health predictions from the server computer 102 and display the received health predictions on the GUI 284. A physician 304 may also use the GUI to enter a prescription and health advices 310 which is sent, via the software network interface 286 through the network 108 and the server computer 102, to the client computing device 110 of a relevant patient 302 for displaying on the GUI 284 thereof.

In embodiments wherein the portable diagnostic devices 104 directly connect to the server computer 102 (via the network 108) without using the diagnostic hub 112, the server computer 102 may comprise the data verification and device calibration module 246 and the data processing module 248.

In some embodiments, the client computing devices 102 of the physicians 304 compile and store all patient-related data such as the diagnostic data, the health predictions, and physician's prescription and health advices as the patient's electronic medical records (EMRs) in the secure database 264.

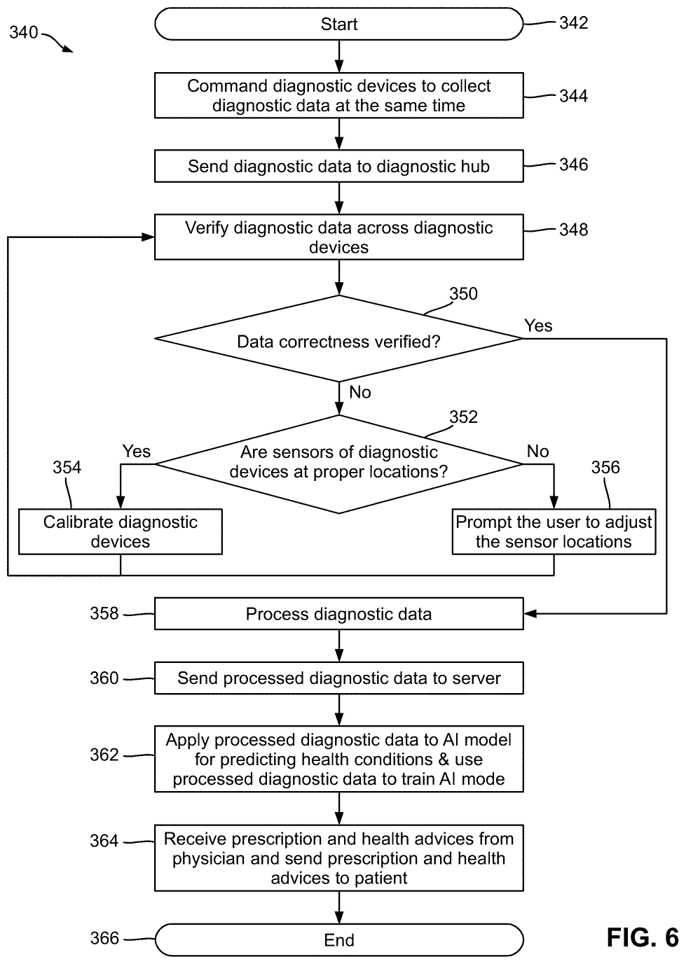

FIG. 6 is a flowchart showing a remote diagnosis and telemedicine process 340 executed by the diagnostic system 100 for performing remote diagnosis and telemedicine, according to some embodiments of this disclosure.

Before the process 340 starts, a patient is required to register an account in the diagnostic system 100 and provide necessary information such as username, password, patient's name, health card number (or the account number in a relevant governing organization or a relevant medical institute), gender, age, height, weight, a brief health history, and/or the like. The information provided by the user is securely transmitted from the patient's client computing device 110 to the server computer 102 and securely stored in the secure database 264 thereof.

Similarly, a physician may also need to register an account in the diagnostic system 100 and provide necessary information such as username, password, physician's name, address, specialty, year of services, availability, and/or the like.

In some embodiments, a patient may designate a physician for diagnosis and health advices. The diagnostic system 100 may rank each registered physician based on patients' reviews.

In some embodiments, diagnostic system 100 may select a physician for a patient based on a set of factors such as the physician's specialty, availability, the type of the medical condition of the patient, and/or the like.

The process 340 starts (step 342) when the patient signs-in to his/her account, attaches sensors of the portable diagnostic devices 104 to various body locations, and provides a START instruction to the diagnostic hub 112 (for example, pressing a START button thereof). Alternatively,

At step 344, the diagnostic hub 112 commands the portable diagnostic devices 104 to start diagnostic data measurements and collect diagnostic data from the sensors thereof. The portable diagnostic devices 104 then transmit collected diagnostic data to the diagnostic hub 112 (step 346). As those skilled in the art will appreciate, different portable diagnostic devices 104 may collect same diagnostic data items (for example, heart rate may be obtained by the electrical stethoscope and heartbeat monitor) and/or different but correlated diagnostic data items (for example, respiratory rate obtained by the heartbeat monitor is correlated with lung sound obtained by the electrical stethoscope 200).

The diagnostic hub 112 verifies the received diagnostic data across all portable diagnostic devices 104 using the combination of the collected diagnostic data (step 348), and checks if the received diagnostic data is correct, for example, whether the diagnostic data collected by a portable diagnostic device 104 is within a predefined range (which may be a predefined range and may be customized automatically or by a physician based on the patient's health conditions) and is consistent with the diagnostic data collected by other portable diagnostic devices 104.

If the data correctness is verified (the “Yes” branch of step 350), the process 340 goes to step 358.

If the diagnostic data item received from a portable diagnostic device 104A is abnormal (the “No” branch of step 350), the diagnostic hub 112 then checks if the sensors of the abnormal portable diagnostic device 104A are at the proper body locations (step 352). For example, if a diagnostic data item received from the abnormal portable diagnostic device 104A is out of the predefined range or the correlation of diagnostic data items received from the abnormal portable diagnostic device 104A and those received from other properly-working portable diagnostic devices 104 is lower than a predefined threshold (that is, diagnostic data items received from the portable diagnostic device 104A are inconsistent with those received from other portable diagnostic devices 104), then the sensors of the portable diagnostic device 104A may be attached to wrong body locations.

If the diagnostic hub 112 determines that the sensors of the abnormal portable diagnostic device 104A are attached to the proper body locations (the “Yes” branch of step 352), the diagnostic hub 112 then calibrate the abnormal portable diagnostic device 104A, for example, using same or correlated diagnostic data items received from other properly-working portable diagnostic devices 104 (step 354). The process 340 then loops back to step 348 to further verify the diagnostic data.

At step 354, the diagnostic hub 112 may determine that the abnormal portable diagnostic device 104A has failed if, after calibration, the diagnostic data item received from the abnormal portable diagnostic device 104A is still abnormal.

If the diagnostic hub 112 determines that the sensors of the abnormal portable diagnostic device 104A are not attached to the proper body locations (the “No” branch of step 352), the diagnostic hub 112 then prompt the patient to adjust the locations of the sensors of the abnormal portable diagnostic device 104A (step 356) for example, by displaying an instruction video clip on the screen of the diagnostic hub 112, playing an instruction sound clip, and/or the like. The process 340 then loops back to step 348 to further verify the diagnostic data.

This loop (step 348 to step 356) may be repeated until a predefined number of loops have reached, at which time the diagnostic hub 112 may provide a feedback (a video clip, a sound clip, and/or the like) to indicate that the patient has failed to attach the sensors of the abnormal portable diagnostic device 104A to the proper body locations and the process 340 may be terminated. On the other hand, the process 340 may exit the loop (step 348 to step 356) when data correctness is verified at step 350. As described above, the process 340 then goes to step 358.

At step 358, the diagnostic hub 112 processes the diagnostic data. For example, the diagnostic hub 112 may combine same data items obtained from different portable diagnostic devices 104 by calculating, for example, a weighted average thereof. Then, the diagnostic hub 112 sends the processed diagnostic data to the server computer 102 (step 360). The combination of diagnostic data may further facilitate the determination of failures in the diagnostic system 100 and the subsequently correction.

At step 362, the AI engine 266 of the server computer 102 applies the processed diagnostic data to the AI model for generating predictions of the patient's health conditions. The AI engine 266 of the server computer 102 also uses the processed diagnostic data to train the AI model. The processed diagnostic data and the generated predictions of the patient's health conditions are stored in the secure database 264 as the patient's EMRs. The trained AI model are stored. Moreover, the processed diagnostic data and the predictions of the patient's health conditions are sent to the client computing device of the patient-designated physician.

At step 364, the physician may review the processed diagnostic data and the predictions of the patient's health conditions and provide a prescription and health advices using the client computing device 110 thereof. The prescription and health advices are transmitted from the physician's client computing device 110 to the server computer 102. The server computer 102 stores the prescription and health advices in the secure database 264 as part of the patient's EMRs, and forwards the prescription and health advices to the client computing device 110 of the patient.

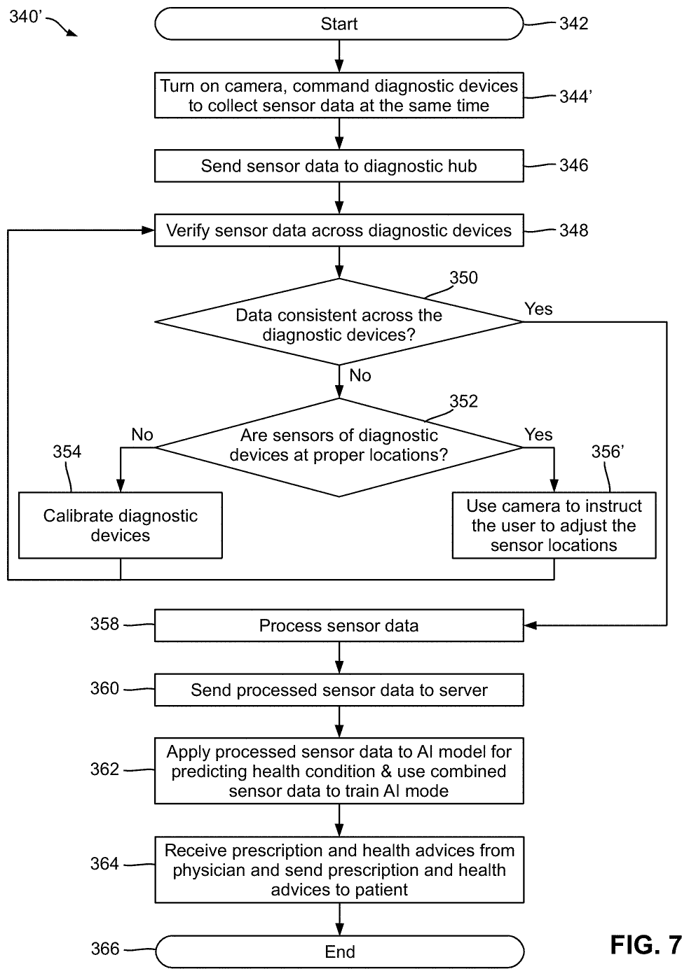

FIG. 7 is a flowchart showing a process 340′ executed by the diagnostic system 100 for performing remote diagnosis and telemedicine, according to some other embodiments of this disclosure.

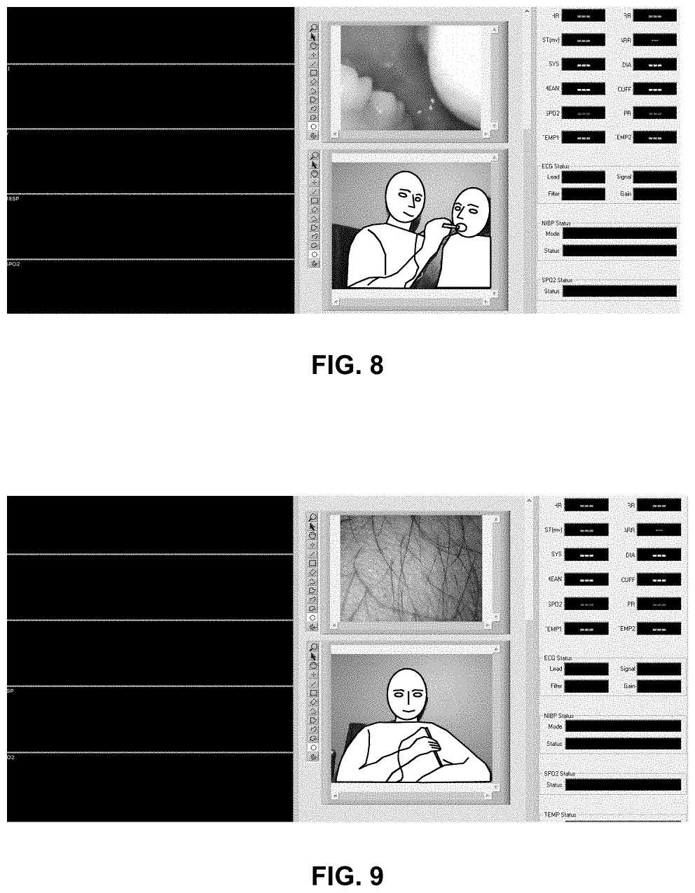

The process 340′ in these embodiments is similar to the process 340 shown in FIG. 6 except that the process 340′ further leverage the cameras in the remote diagnosis. More specifically, the process 340′ modifies process 340 in the following steps:
step 344′ (corresponding to step 344 of the process 340): at step 344′, the camera on the patient side is turned on and a video communication is established between the camera on the patient side and a camera on the patient-designated physician side (and a corresponding audio communication is also established) to allow the physician to conduct visual diagnosis and to help the patient to position the sensors of the portable diagnostic devices 104 (for example, see FIGS. 8 and 9), and in some embodiments, to allow the physician to remotely start the operation of the portable diagnostic devices 104 when the sensors thereof are at proper positions; and
step 356′ (corresponding to step 356 of the process 340): at step 356′ wherein one or more sensors of the portable diagnostic devices 104 are not positioned at proper body locations, the physician may use the video communication to check the sensor locations and use the audio/video communication to instruct the patient how to position the sensors.

By using the camera on the patient side, the AI engine 266 may be used by to analyze images captured by the camera at step 344′ using machine learning while preserving privacy, for determining potential medical issues and predicting potential disease in various areas such as nasal, ears, throat, skins, and/or the like.

The diagnostic system 100 disclosed herein leverages various portable diagnostic devices 104 to provide a low cost solution for patient-physician interaction. The patient may conveniently use the portable diagnostic devices 104 at home, outdoor, in travel, at healthcare community, hospitals, urgent care centers (UCCs), intensive care units (ICUs) to continuously monitor the vital clinical parameters of the patient such as NIBP, ECG, SpO₂, respiration, body temperature, stethoscope, otoscope, and/or the like. By using sensors that may be individually positioned at various body locations most suitable for capturing diagnostic data, the accuracy of the collected clinical parameters of the patient is easily maintained.

The vital clinical parameters of the patient may be transmitted to the patient-designated physician in real-time to allow the physician to evaluate the patient's health conditions in a prompt and comprehensive manner.

By using the diagnostic hub 112, the cost of the portable diagnostic devices 104 may be further lowered by eliminating the hardware and software network interface therefrom. Moreover, the diagnostic hub 112 may display the collected diagnostic data to the patient on the screen thereof in a readable form, thereby allowing the patient to monitor his/her health conditions when the physician is offline.

In some embodiments, the diagnostic hub 112 may also store the collected diagnostic data on a memory or storage device thereof.

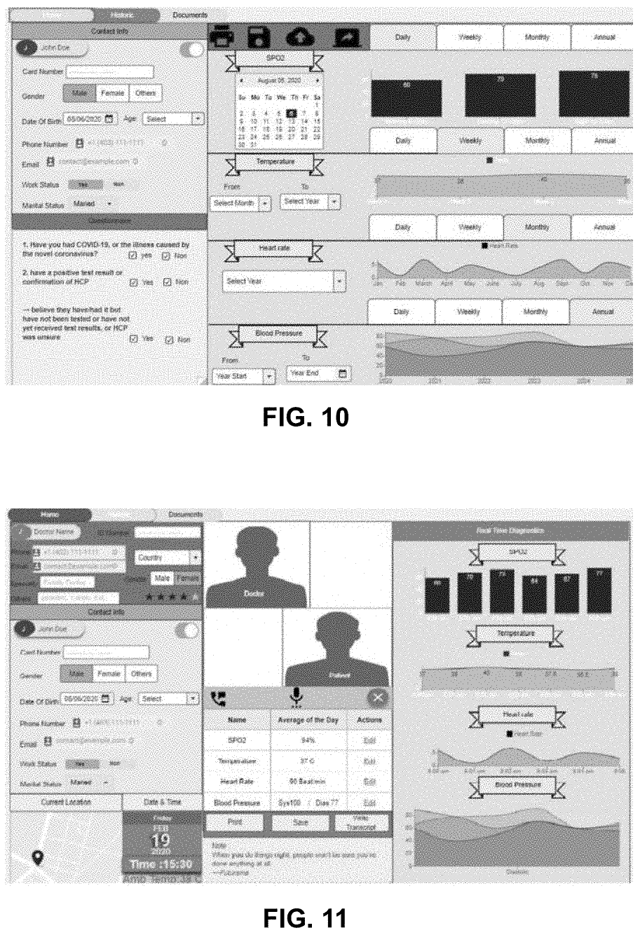

In some embodiments, the diagnostic hub 112 may be interconnected with the patient's client computing device 110 via a suitable wired or wireless communication technology such as USB or BLUETOOTH® for exchanging data (for example, transferring the collected diagnostic data) and analyzing diagnostic data, and/or displaying processed diagnostic data and/or the analytical results thereof. For example, FIG. 10 shows an exemplary patient's view displayed on the patient's client computing device 110. FIG. 11 shows an exemplary physician's view displayed on the physician's client computing device 110 for displaying processed diagnostic data and/or the analytical results thereof.

In various embodiments, the diagnostic system 100 disclosed herein provides the following functionalities:
teleconferencing between the patient and the physician;
real-time bio-signal reading and display sharing between the patient and the physician;
controlling the portable diagnostic devices 104 and the diagnostic hub 112 by either the patient or the physician; and
recording diagnostic data and other patient-related data in secure storage on the server computer 102, the client computing device 110 of the patient, and/or the client computing device 110 of the physician.

In some embodiments, the diagnostic system 100 disclosed herein may also provide training of medical students by providing them with selected access to the diagnostic data stored in the secure database 264 and/or allowing them to attend selected remote diagnosis and telemedicine processes.

Thus, compared to conventional remote healthcare monitoring and telemedicine systems, the diagnostic system 100 disclosed herein provides various advantages such as:
real-time and continuous collection of patients' diagnostic data as needed;
real-time and continuous communication of patients' diagnostic data from the portable diagnostic devices 104 to the server computer 102 and the physicians' client computing devices 110;
detection and correction of sensor mis-positioning;
improved stethoscope having a stethoscope sensor on a belt which provides the ease of self-positioning of the stethoscope sensor at proper location about the patient's body for diagnosis;
improved accuracy of the collected diagnostic data by positioning sensors at most suitable body locations, collecting diagnostic data from various sensors simultaneously, and by combing collected diagnostic data;
fast and efficient diagnosis through real-time data and audio/video communication;
improved diagnosis by using AI and machine learning;
The use of the portable diagnostic devices 104 and the diagnostic hub 112 providing the patient with mobility, ease of use, and diagnostic data accuracy;
a low-cost and convenient solution for patients, healthcare communities, hospitals, UCCs, ICUs, and/or the like with reduced waiting time, reduced travel time (for patients to visit physicians and/or for physicians to visit patients), and improved health system efficiency;
helping elderlies, patients in remote areas, and patients with disabilities in their healthcare needs without meeting physicians in person;
improved safety against respiratory diseases (such as flue, SAS, MERS, COVD-19, and/or the like) and other diseases by reducing face-to-face patient-physician interaction.

Thus, the diagnostic system 100 disclosed herein may help in solving various issues in conventional healthcare systems such as:
High cost of healthcare systems;
Growing cost of healthcare systems;
Long waiting time of patients;
Lack of healthcare workers;
Long working hours of healthcare workers;
Increased needs of healthcare services;
Inadequate infrastructure in hospitals and mostly in rural or remote areas;
Low-availability of family doctors;
Lack of adequate opportunities for training or continuing medical education for doctors in rural areas or for women in some cases.

In above embodiments, the diagnostic hub 112 processes sensor data at step 358 and sends the processed diagnostic data to the server computer 102 at step 360. In some alternative embodiments, the diagnostic hub 112 sends unprocessed diagnostic data (for example, the diagnostic data collected at step 346) to the server computer 102. The server computer 102 then processes sensor data (similar as step 358).

In some embodiments, the AI engine 266 may also be used data verification and device calibration based on the diagnostic data received by the server computer 102.

In some embodiments, the diagnostic system 100 may not comprise the server computer 102 or the server computer 102 does not run the AI engine 266. In these embodiments, each diagnostic hub 112 runs an AI engine 266 for applying the processed diagnostic data to an AI model for generating predictions 308 of patients' health conditions. In some related embodiments, the diagnostic hubs 112 may be directed interconnected with each other via suitable wired or wireless communication technologies. In these embodiments, the diagnostic hubs 112 may exchange the processed diagnostic data of different patients and/or the AI models of different patients so that each AI engine 266 of these diagnostic hubs 112 may combine other patient's diagnostic data (for example, the blood glucose data of other people in a family) for generating a more precise prediction 308 of its patient's health conditions (such as some genetic diseases or hereditary diseases).

Although embodiments have been described above with reference to the accompanying drawings, those of skill in the art will appreciate that variations and modifications may be made without departing from the scope thereof as defined by the appended claims.

What is claimed is:

1. A system comprising:
a plurality of diagnostic sensors; and
a first computing device interconnected to the plurality of diagnostic sensors for:
commanding the plurality of diagnostic sensors to collect diagnostic data from a patient;
determining if each of the plurality of diagnostic sensors are positioned at respectively predefined body locations of the patient;
combining the collected diagnostic data for analysis when all of the plurality of diagnostic sensors are positioned at respectively predefined body locations of the patient; and
prompting the patient to reposition at least one of the plurality of diagnostic sensors when the at least one sensor is determined as being mis-positioned from the corresponding predefined body location.

2. The system of claim 1 further comprising a camera, wherein the first computing device is interconnected with the camera for audio and video communication with the patient.

3. The system of claim 1, wherein the first computing device is configured to calibrate the plurality of diagnostic sensors.

4. The system of claim 1, wherein the first computing device is configured to verify the collected diagnostic data.

5. The system of claim 1, wherein the plurality of diagnostic sensors comprises one or more of a blood pressure monitor, an oximeter, a temperature sensor, an electrical stethoscope, a heart-beat monitor and a high-definition otoscope.

6. The system of claim 1 further comprising an ambient temperature sensor for measuring ambient temperature proximate the patient.

7. The system of claim 1, further comprising one or more positioning components to determine a geographical location of the first computing device.

8. The system of claim 1, wherein the first computing device is further configured for:
sending at least one of the collected diagnostic data and the combined diagnostic data to a second computing device via a network.

9. The system of claim 8, wherein said sending the at least one of the collected diagnostic data and the combined diagnostic data to the second computing device via the network comprises:
sending in real-time the at least one of the collected diagnostic data and the combined diagnostic data to the second computing device via the network.

10. The system of claim 8, wherein the second computing device is configured for processing the collected diagnostic data and the combined diagnostic data into processed diagnostic data, and the second computing device comprises an artificial intelligence (AI) model for predicting health conditions of the patient using the processed diagnostic data.

11. A method of remote medical diagnosis comprising the steps of:
collecting diagnostic data from a patient using a plurality of diagnostic sensors;
determining if all of the plurality of diagnostic sensors are positioned at respectively predefined body locations of the patient;
combining the collected diagnostic data for analysis when all of the plurality of diagnostic sensors are positioned at respectively predefined body locations of the patient; and
prompting the patient to reposition at least one of the plurality of diagnostic sensors when the at least one sensor is determined as being mis-positioned from the corresponding predefined body location.

12. The method of claim 11 further comprising the step of turning on a camera, and wherein the step of prompting the patient comprises communicating with the patient with audio and video.

13. The method of claim 11 further comprising the step of calibrating at least one of the diagnostic sensors.

14. The method of claim 11 further comprising the step of verifying the diagnostic data.

15. The method of claim 11, wherein the step of collecting diagnostic data comprises one or more of collecting a non-invasive blood pressure, collecting a peripheral oxygen saturation level, collecting a body temperature, collecting an electrical stethoscope measurement, collecting an electrocardiogram, and collecting a high-definition otoscope reading.

16. The method of claim 11 further comprising the step of measuring ambient temperature of the patient's environment.

17. The method of claim 11 further comprising the step of determining a geographical location of the system.

18. The method of claim 11 further comprising the step of:
sending at least one of the collected diagnostic data and the combined diagnostic data to a computing device via a network.

19. The method of claim 18, wherein the step of sending the collected diagnostic data comprises sending in real-time the at least one of the collected diagnostic data and the combined diagnostic data to the computing device via the network.

20. The method of claim 18 further comprising the steps of:
processing the collected diagnostic data and the combined diagnostic data into processed diagnostic data; and
predicting health conditions of the patient using an AI model and the processed diagnostic data.
